(19) United States
(12) Patent Application Publication
(10) Pub. No.: US 2024/0082263 A1
(43) Pub. Date: Mar. 14, 2024

(54) PHARMACEUTICAL COMPOSITION FOR COMBINATION THERAPY COMPRISING MELATONIN AND PROSTAGLANDIN E2 FOR TREATING INTESTINAL EPITHELIAL INJURY AS AN ACTIVE INGREDIENT

(71) Applicant: PUSAN NATIONAL UNIVERSITY INDUSTRY-UNIVERSITY COOPERATION FOUNDATION, Busan (KR)

(72) Inventors: Hyung-Sik KIM, Yangsan-si (KR); Yoo Jin SEO, Yangsan-si (KR); Ji-Su AHN, Yangsan-si (KR)

(73) Assignee: PUSAN NATIONAL UNIVERSITY INDUSTRY-UNIVERSITY COOPERATION FOUNDATION, Busan (KR)

(21) Appl. No.: 18/144,230

(22) Filed: May 7, 2023

(30) Foreign Application Priority Data
May 10, 2022 (KR) 10-2022-0057104

(51) Int. Cl. A61K 31/559; A61K 31/165; A61K 31/4045; A61K 31/5575; A61P 1/00

(52) U.S. Cl. CPC A61K 31/559; A61K 31/165; A61K 31/4045; A61K 31/5575; A61P 1/00

(57) ABSTRACT

The present invention relates to a pharmaceutical composition for combined administration of melatonin and prostaglandin E₂ (PGE₂) for the treatment of intestinal epithelial injury diseases, and it is confirmed that when intestinal epithelial organoids are treated in combination with melatonin and PGE₂, the expression of revival stem cell markers increases more than when the two substances are treated alone, and therefore it can be usefully used as a composition for preventing, treating or improving intestinal epithelial injury diseases.

### TECHNICAL FIELD

The present invention relates to a pharmaceutical composition for combined administration of melatonin and prostaglandin E2 for treating intestinal epithelial injury.

### BACKGROUND ART

Inflammatory bowel disease, represented by ulcerative colitis and Crohn's disease, is a chronic and recurrent inflammatory disease of the stomach of unknown cause. It is a rare and intractable disease in which symptoms such as diarrhea, bloody stool, abdominal pain, weight loss, and severe fatigue occur continuously for weeks or months and are accompanied by serious complications such as stricture or perforation. As of 2019, there are 4 million patients with inflammatory bowel disease worldwide, and the morbidity rate is steadily increasing due to changes in eating habits, income levels, and improved hygiene. In particular, South Korea has the highest prevalence rate in Asia, and the economic population in their 20 s and 30 s has a high morbidity rate, so the socioeconomic loss is very large.

Since the etiology of inflammatory bowel disease has not been clearly identified, maintenance of remission in which clinical symptoms are controlled below a certain level is an important treatment goal, and therapeutic agents having an Immunomodulatory or anti-inflammatory effect have been mainly used. However, the first-line drugs such as 5-aminosalicylic acid, steroids, and immunosuppressants have problems such as refractory, tolerance, and side effects, and there are also many patients who show refractory to the recently emerging immune response suppression-based biological formulations. As side effects caused by extensive immunosuppression, such as opportunistic infections, tuberculosis, and lymphoma, have been reported, there is a demand for the development of therapeutic agents with new mechanisms. In particular, in order to induce mucosal healing, which is the ultimate treatment goal of inflammatory bowel disease, a treatment strategy capable of maximizing the regeneration of the intestinal epithelium itself is required.

Intestinal tissue with the highest cell turnover rate in the body has superior regenerative ability due to interaction between intestinal stem cells/stem cell microenvironment (Niche) and high cellular plasticity. In normal intestinal tissue, Lgr5-expressing intestinal stem cells present in the crypt of the intestinal epithelial layer are responsible for maintaining homeostasis, but it has been found that they are extremely vulnerable to damage and are rapidly lost after damage, so studies have been conducted to identify cell populations specialized in regeneration processes. In particular, intestinal organoids with a well-reproduced intestinal epithelial structure have the advantages of a shorter culture period than other organ organoids, feasibility of in vitro propagation and storage and cultivation, and similar physiological and chemical properties to external stimuli as observed in vivo, as well as easy experimental manipulation, so it is actively used in related studies.

Recently, it has been reported that a new stem cell population, revival stem cells (RSCs), can be induced to completely reconstitute the intestinal epithelial tissue during the process of regenerating injured intestinal tissue through mouse model studies. These cells have clusterin and Ly6a as specific markers, and when PGE2 (Prostaglandin E2) secreted from fibroblasts in the intestinal mucosa in response to damage caused by irradiation or administration of Dextran Sulfate Sodium (DSS), a substance that damages the intestinal epithelium, acts on the EP4 receptor, one of the PGE2-specific receptors present in intestinal epithelial cells, the YAP signaling system is activated. The results that clusterin-deficient mice or inhibition of PGE2-EP4 interaction or YAP activation significantly reduced the injury recovery suggests that the induction of these revival stem cells plays a key role in intestinal epithelial regeneration. In this regard, there is a study showing that when epigenetic regulators VPA, inhibitor of histone deacetylase, and EPZ6438, an enhancer of Zeste 2 Polycomb Repressive Complex 2 (EZH2), were administered together to an intestinal epithelial organoid or a DSS-mediated inflammatory bowel disease model, a revival stem cell population is induced, thereby exhibiting a therapeutic effect. However, epigenetic modulators have a great weakness in terms of safety because they have a high possibility of causing non-specific gene expression and suppression. Therefore, there is a need to find a new combination of bioactive substances with guaranteed safety in order to induce revival stem cells in the intestinal epithelium.

In addition, PGE2, which is known to play a key role in inducing revival stem cells, can have a wide range of physiological activities because it targets not only intestinal epithelial cells but also various target cells such as immune cells and has various types of receptors. Therefore, in order to safely and effectively achieve epithelial regeneration through the induction of revival stem cells, there is a need to discover receptor-specific inducers that can minimize non-specific reactions and optimize the timing of RSC induction.

### PRIOR ART DOCUMENT

### Patent Documents

(Patent document 1) Chinese Patent Publication CN 112516169 (published on Mar. 19, 2021)

### DISCLOSURE

### Technical Problem

An object of the present invention is to provide a pharmaceutical composition for combined administration of melatonin and PGE2 (prostaglandin E2) for treating intestinal epithelial injury diseases.

### Technical Solution

In order to achieve the above object, the present invention provides a pharmaceutical composition for preventing or treating intestinal epithelial injury disease comprising melatonin or an agonist of melatonin receptor; and PGE2 (prostaglandin E2) or an agonist of PGE2 receptor as an active ingredient.

Also, the present invention provides a health functional food composition for preventing or improving intestinal epithelial injury disease comprising melatonin or an agonist of melatonin receptor; and PGE2 (prostaglandin E2) or an agonist of PGE2 receptor as an active ingredient.

In addition, the present invention provides a composition for inducing revival stem cells comprising melatonin or an agonist of melatonin receptor; and PGE2 (prostaglandin E2) or an agonist of PGE2 receptor as an active ingredient.

In addition, the present invention provides a pharmaceutical composition for combined administration for treating intestinal epithelial injury disease comprising melatonin or an agonist of melatonin receptor; and PGE2 (prostaglandin E2) or an agonist of PGE2 receptor as an active ingredient.

In addition, the present invention provides a method of treating intestinal epithelial injury diseases comprising administering melatonin or an agonist of melatonin receptor; and PGE2 (prostaglandin E2) or an agonist of PGE2 receptor as an active ingredient.

Furthermore, the present invention provides a method of inducing revival stem cells comprising administering melatonin or an agonist of melatonin receptor; and PGE2 (prostaglandin E2) or an agonist of PGE2 receptor as an active ingredient.

### Effects of the Invention

According to the present invention, it is confirmed that when intestinal epithelial organoids are treated in combination with melatonin and PGE2, the expression of revival stem cell markers increases more than when the two substances are treated alone, and therefore it can be usefully used as a composition for preventing, treating or improving intestinal epithelial injury diseases.

### DESCRIPTION OF DRAWINGS

FIGS. 1a and 1b shows a result of comparing and analyzing the expression and cell induction changes of Ly6a, a revival stem cell-specific marker, in mouse intestinal epithelial organoids according to melatonin and PGE₂ treatment.

FIG. 2 shows a result of comparing and analyzing the changes of Ly6a expression in mouse intestinal epithelial organoids through activation of MT1, MT2, EP2, and EP4 receptors. For the receptor activation, the following materials were used: 2-iodomelatonin was used for MT1 receptor activation, 8M-PDOT for MT2 receptor activation, Butaprost for EP2 receptor activation, and Cay10598 for EP4 receptor activation.

FIG. 3 shows a result of comparing and analyzing the expression changes of Clusterin, a revival stem cell marker, according to treatment with melatonin, 8M-PDOT, and PGE₂ in human-derived intestinal epithelial organoids.

### DETAILED DESCRIPTION OF THE INVENTION

Hereinafter, the present invention will be described in more detail.

The present invention provides a pharmaceutical composition for preventing or treating intestinal epithelial injury disease comprising melatonin or an agonist of melatonin receptor; and PGE₂ (prostaglandin E2) or an agonist of PGE₂ receptor as an active ingredient.

The agonist of the melatonin receptor may be 8M-PDOT.

The PGE₂ receptor agonist may be at least one selected from the group consisting of Cay10598, TCS2510, L-902,688 and ONO-AEI-329.

The pharmaceutical composition may contain 100 to 600 μM of melatonin or its receptor agonist and 0.05 to 20 μM of PGE₂ or its receptor agonist, but it is not limited thereto.

In addition, the pharmaceutical composition is characterized by exhibiting an effect of inducing revival stem cells.

In addition, the pharmaceutical composition may increase the expression of one or more markers selected from the group consisting of clusterin, Ly6a and Claudin-4, but it is not limited thereto.

The intestinal epithelial injury disease is at least one selected from the group consisting intestinal metaplasia, Crohn's disease, ulcerative colitis, ulcerative duodenitis, hemorrhagic rectal ulcer, leaky gut syndrome, gastritis, gastric ulcer, pouchitis, enteritis and ischemic colitis, but it is not limited thereto.

The pharmaceutical composition of the present invention can be prepared in a unit dose form or in a multi-dose container by formulating using a pharmaceutically acceptable carrier according to a method that can be easily performed by those skilled in the art to which the invention pertains.

The pharmaceutically acceptable carrier is one commonly used in formulation, and includes lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate and mineral oil, and the like, but it is not limited thereto. The pharmaceutical composition of the present invention may further include lubricants, wetting agents, sweeteners, flavoring agents, emulsifiers, suspending agents, preservatives, and the like, in addition to the above components.

In the present invention, the content of the additives included in the pharmaceutical composition is not particularly limited and may be appropriately adjusted within the range of content used in conventional formulations.

The pharmaceutical composition may be formulated into the form of one or more external agents selected from the group consisting of an injectable formulation such as aqueous solution, suspension, or emulsion, pill, capsule, granule, tablet, cream, gel, patch, spray, ointment, plaster, lotion, liniment, pasta, and cataplasma.

The pharmaceutical composition of the present invention may additionally contain pharmaceutically acceptable carriers and diluents for formulation. The pharmaceutically acceptable carriers and diluents include excipients such as starch, sugar and mannitol, fillers and extenders such as calcium phosphate, binder such as cellulose derivatives including carboxymethyl cellulose and hydroxypropyl cellulose, gelatin, alginates, and polyvinyl pyrrolidone, etc., lubricants such as talc, calcium stearate, hydrogenated castor oil and polyethylene glycol, disintegrants such as povidone, crospovidone, surfactants such as polysorbates, cetyl alcohol, and glycerol, but it is not limited thereto. The pharmaceutically acceptable carrier and diluent may be biologically and physiologically compatible with the subject. Examples of diluents include saline, aqueous buffers, solvents and/or dispersion media, but they are not limited thereto.

The pharmaceutical composition of the present invention may be administered orally or parenterally (e.g., intravenous, subcutaneous, intraperitoneal or topical application) depending on the desired method. For oral administration, it may be formulated into tablets, troches, lozenges, aqueous suspensions, oily suspensions, powders, granules, emulsions, hard capsules, soft capsules, syrups or elixirs, etc. For parenteral administration, it may be formulated into injection solution, suppository, powder for respiratory inhalation, aerosol for spray, ointment, powder for application, oil or cream, etc.

The dosage of the pharmaceutical composition of the present invention depends on the condition and weight, age, sex, health condition, dietary constitution specificity of the patient, the nature of the preparation, the severity of the disease, the administration time of the composition, the administration method, the administration period or interval, the excretion rate and the type of drug, which may be appropriately selected by a person skilled in the art. For example, it may range from about 0.1 to 10,000 mg/kg, but it is not limited thereto, and may be divided and administered once or several times a day.

The pharmaceutical composition may be administered orally or parenterally (e.g., intravenous, subcutaneous, intraperitoneal or topical application) depending on the desired method. The pharmaceutically effective amount and effective dose of the pharmaceutical composition of the present invention may vary depending on the formulation method, administration method, administration time and/or administration route of the pharmaceutical composition, and a person of ordinary skill in the art can readily determine and prescribe an effective dosage for the desired treatment. Administration of the pharmaceutical composition of the present invention may be administered once a day, or may be divided and administered several times.

In addition, the present invention provides a health functional food composition for preventing or improving intestinal epithelial injury disease comprising melatonin or an agonist of melatonin receptor; and PGE₂ (prostaglandin E2) or an agonist of PGE₂ receptor as an active ingredient.

The present invention can be generally used as a commonly used food.

The food composition of the present invention can be used as a health functional food. The term “health functional food” refers to food manufactured and processed using raw materials or ingredients having useful functionalities for the human body in accordance with the Health Functional Foods Act, and the term “functional” refers to intake for the purpose of obtaining useful effects for health purposes such as adjusting nutrients for the structure and function of the human body or physiological functions.

The health functional food composition may include conventional food additives, and the suitability as the “food additive” is determined by the specification and standards for the applicable item in accordance with General Regulations and General Test Methods of Korean Food Additives Codex approved by the Ministry of Food and Drug Safety, unless otherwise provided.

Examples of the items published in the above-mentioned “Korean Food Additives Codex” include chemical synthetics such as ketones, glycine, potassium citrate, nicotinic acid, and cinnamic acid and the like, natural additives such as persimmon color, licorice extract, crystalline cellulose, kaoliang color and guar gum and the like, mixed preparations such as L-sodium glutamate preparation, alkaline agents for noodles, preservative formulation and a tar color formulation and the like.

The food composition of the present invention may be prepared and processed in the form of tablets, capsules, powders, granules, liquids, pills and the like. For example, among health functional foods in the form of capsules, hard capsules can be prepared by mixing and filling a composition according to the present invention with additives such as excipients in a conventional hard capsule, and soft capsules can be prepared by mixing the composition according to the present invention with additives such as excipients and filling them in a capsule base such as gelatin. The soft capsule may contain a plasticizer such as glycerin or sorbitol, a coloring agent, a preservative, and the like, if necessary.

Definitions of terms for the excipients, binders, disintegrants, lubricants, bitters, flavoring agents, etc. are described in literature known in the art, and include those having the same or similar functions. There is no particular limitation on the type of food, and it includes all health functional foods in a conventional sense.

As used herein, the term “prevention” refers to any action of inhibiting or delaying a disease by administering the composition according to the present invention. As used herein, the term “treatment” refers to any action that improves or beneficially alters the symptoms of a disease by administering the composition according to the present invention. As used herein, “improvement” means any action that improves the bad condition of a disease by administering or ingesting the composition of the present invention to a subject.

In addition, the present invention provides a composition for inducing revival stem cells comprising melatonin or an agonist of melatonin receptor; and PGE₂ (prostaglandin E2) or an agonist of PGE₂ receptor as an active ingredient.

In addition, the present invention provides a pharmaceutical composition for combined administration for treating intestinal epithelial injury disease comprising melatonin or an agonist of melatonin receptor; and PGE₂ (prostaglandin E2) or an agonist of PGE₂ receptor as an active ingredient.

In addition, the present invention provides a method of treating intestinal epithelial injury diseases comprising administering melatonin or an agonist of melatonin receptor; and PGE₂ (prostaglandin E2) or an agonist of PGE₂ receptor as an active ingredient.

Furthermore, the present invention provides a method of inducing revival stem cells comprising administering melatonin or an agonist of melatonin receptor; and PGE₂ (prostaglandin E2) or an agonist of PGE₂ receptor as an active ingredient.

Hereinafter, the present invention will be described in more detail through examples. These examples are intended to explain the present invention in more detail, and the scope of the present invention is not limited to these examples.

### [EXAMPLE 1] Confirmation of Induction of Revival Stem Cell Population by Treatment of Melatonin and PGE₂ in Mouse Intestinal Epithelial Organoids

Mouse-derived small intestinal epithelial organoids were obtained as follows. First, small intestine tissue (duodenum about 10-15 cm) was isolated from 8-week-old C57BL/6N mice, and the intestinal lumen was washed with PBS using a 1 ml pipette tip, and the tissue was opened with the lumen facing up, and the remaining mucus, food, and intestinal villi were scraped off and removed. Thereafter, about 25 ml of PBS was filled in a 50 ml tube, and the intestinal tissue was cut to a length of about 2 to 4 mm and washed by shaking vigorously (repeated about 3 to 4 times). The washed intestinal tissue was suspended in 20 ml of Gentle cell dissociation reagent (100-0485, stemcell technologies) and reacted for 15 to 20 minutes while shaking at 20 rpm at room temperature. After the reaction was completed, crypts were separated from the intestinal tissue by vortexing at maximum speed for 5 seconds, and the tissue was removed and centrifuged at 290 g for 5 minutes. The collected cell pellet was resuspended in 10 ml of PBS, centrifuged at 290 g for 5 minutes, and then the cell pellet was suspended in DMEM/F12 medium to confirm the number of crypts. About 500 crypts per 24 well are mixed with 20 μl of cell culture medium (IntestiCult™ Organoid Growth Medium for Mouse, 05005, stemcell technologies) and 20 μl of Matrigel (354230, corning), planted in a dome shape in the center of the well, and placed in a cell culture incubator. After standing for 10 minutes and waiting for the gel to harden, 750 μl of IntestiCult was added and cultured.

The obtained mouse-derived small intestinal epithelial organoids were treated with melatonin (500 μm, 14427, Cayman) and PGE₂ (prostaglandin E2, 100 nM; 14010, Cayman) alone or in combination for 5 days, and microarray was performed to track gene expression changes. RNA was extracted from the organoids using the RNeasy Kit (Qiagen), cDNA was synthesized using the GeneChip WT (Whole Transcript) Amplification kit, and analyzed using the Clariom™ S Affymetrix GeneChip Array (Affymetrix).

As a result of comparing the fold change by calculating the expression difference between each group, PGE₂ induced Clusterin (hereinafter referred to as Clu) and Ly6a, which are revival stem cell markers and Claudin-4 (hereinafter referred to as Cldn4), which is an epithelial regeneration-related marker, and showed the most excellent induction effect, especially when combined with melatonin (Table 1).

TABLE 1

| | M/C | M/C | P/C | P/C | MP/C | MP/C |
|---|---|---|---|---|---|---|
| | FC | p-value | FC | p-value | FC | p-value |
| Cldn4 | 1.0013 | 0.9912 | 1.9264 | 0.0559 | 3.9237 | 0.0101 |
| Clu | 1.2252 | 0.6539 | 1.7590 | 0.1632 | 8.7925 | 0.0488 |
| Ly6a | 1.1646 | 0.4268 | 9.7349 | 0.0158 | 45.3127 | 0.0172 |

FC = fold change
M/C = melatonin/control
P/C = PGE₂/control
MP/C = melatonin-PGE₂/control

In addition, changes in the percentage of cells expressing Ly6a, a specific marker for revival stem cells, were evaluated by flow cytometry. For flow cytometry, mouse small intestine epithelial organoids were treated with melatonin (500 μM) and PGE₂ (100 nM) alone or in combination for 5 days, and the organoids were reacted with TrypLE™ Express Enzyme (Thermo Fisher) for 5 minutes at 37° C. to produce single cells. Thereafter, the Ly6a antibody (12-5981-82, Thermo Fisher) fluorescently labeled with PE was diluted 1:250 in PBS containing 3% BSA, and 200 μl of the diluted solution was mixed with the single celled sample and allowed to stand in a dark room at 4° C. for 1 hour. Then, each sample was washed with PBS (300 g, centrifuged for 5 minutes) and immediately analyzed with an Accuri C6 flow cytometer.

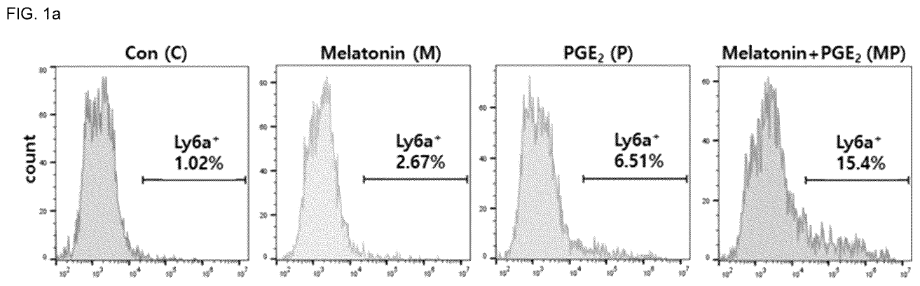

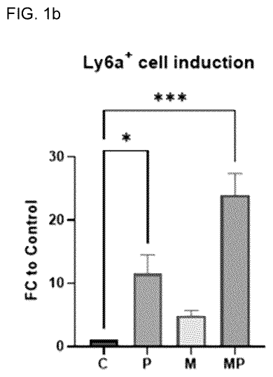

As a result, it was confirmed that Ly6a expression was most remarkably increased upon melatonin-PGE₂ combined treatment (FIG. 1a and FIG. 1b).

### [EXAMPLE 2] Confirmation of Induction of Revival Stem Cell Population by Treatment of Mouse Intestinal Epithelial Organoid with Melatonin Analogue and PGE₂ Receptor Specific Agonist

In order to further specify the mechanism of inducing revival stem cells, mouse-derived intestinal epithelial organoids were treated with a combination of various melatonin analogues and a specific agonist of the PGE₂ receptor. As melatonin analogues, 2-iodomelatonin (200 μM; 19711, Cayman) or 8M-PDOT (200 μM; 29521, Cayman), specific agonist for MT1 or MT2 receptors, which are representative receptors of melatonin, were used, and as PGE₂ analogues, Butaprost (10 μM; 13740, Cayman) and Cay10598 (10 μM; 13281, Cayman), specific analogues of EP2 and EP4 receptors, respectively, were used. After treatment with each combination for 5 days, flow cytometric analysis was performed on the ratio of Ly6a-expressing cells in the same manner as in Example 1.

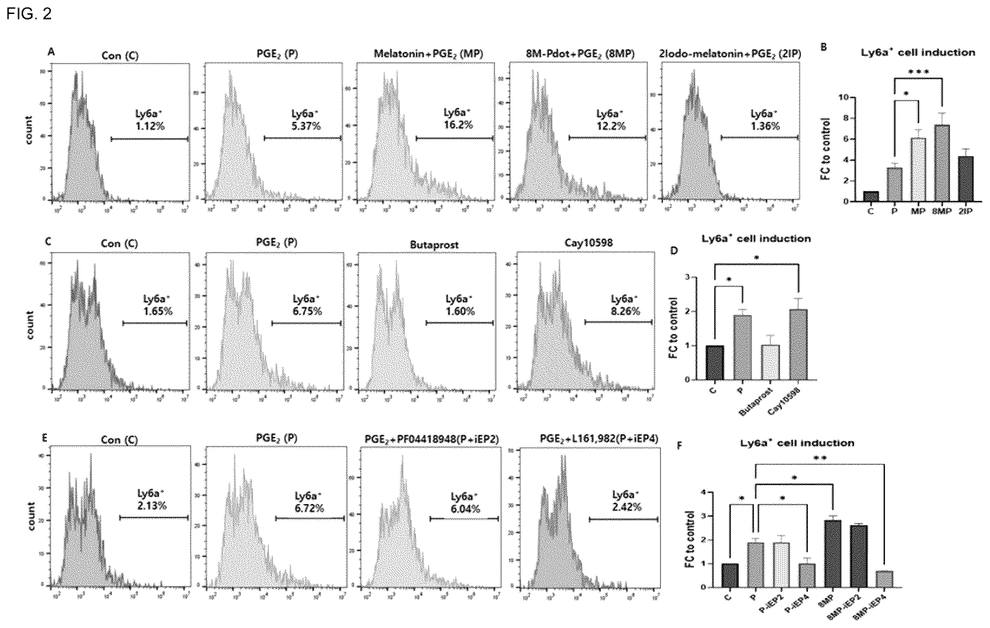

As a result, the combination of PGE₂ and 8M-PDOT showed a similar degree of revival stem cell induction ability as the combination of PGE₂ and melatonin, whereas the combination of PGE₂ and 2-iodomelatonin did not show a specific induction effect. In addition, as a result of treatment with Butaprost and Cay10598, Butaprost did not show a specific induction effect, whereas Cay10598 showed an induction effect similar to or higher than that of PGE₂ (FIG. 2). This suggests that revival stem cells are induced when the EP4 receptor is specifically activated.

To verify this hypothesis, the EP4 receptor-specific inhibitor L-161,982 (10 μM; 1001156, Cayman) was co-treated to the PGE₂ alone treatment group and the 8M-PDOT and PGE₂ combined treatment group. It was confirmed that the induction of revival stem cells was greatly suppressed by the treatment of L-161,982. In contrary, there was no effect when treated with PF-04418948 (20 μM, 15016, Cayman), an EP2 receptor-specific inhibitor (FIG. 2).

In conclusion, it was demonstrated that the revival stem cells are induced by the EP4 receptor activation and the synergistic action by melatonin is mediated by the MT2 receptor activation.

### [EXAMPLE 3] Confirmation of Induction of Revival Stem Cell Population by Treatment of Melatonin and PGE₂ Receptor Specific Agonists in Human Intestinal Epithelial Organoids

Human-derived intestinal epithelial organoids were treated with melatonin and its analogues and PGE₂ to confirm whether revival stem cells were induced as observed in mouse intestinal organoids. In order to obtain intestinal epithelial organoids, donated human colon tissue was cut into a size of about 1 cm 2, placed in PBS containing gentamicin (10 μg/ml, Gibco), and allowed to stand at 4° C. for 30 minutes. Then, the tissue was taken out and the mucous layer was scraped off using forceps, and the process of shaking and washing in PBS was repeated 5 to 6 times, as in the case of mice. After washing, the tissue was put into 25 ml of Gentle cell dissociation reagent and reacted by shaking at 200 rpm for 20 minutes at 37° C. After vortexing at the maximum speed, the crypts were separated and cultured by washing in the same process as in mice. At this time, IntestiCult™ Organoid Growth Medium for Human (06010, stemcell technologies) was used as the culture medium. The obtained human intestinal epithelial organoids were treated alone or in combination with melatonin, 8M-PDOT, and PGE₂ at the same concentrations as in the mouse experiment, and then the expression level of Clusterin (CLU) was compared through quantitative PCR. RNA was extracted with the RNeasy Kit and cDNA was synthesized, followed by reaction at 95° C. for 10 seconds; and 60° C. for 30 seconds (40 cycles) to perform the experiment. The CLU primers used at this time were Forward: 5′-GTTGCTTTTGCACCTACGGG-3′ and Reverse: 5′-GAGCAGCAGAGTCGAGTGTT-3′.

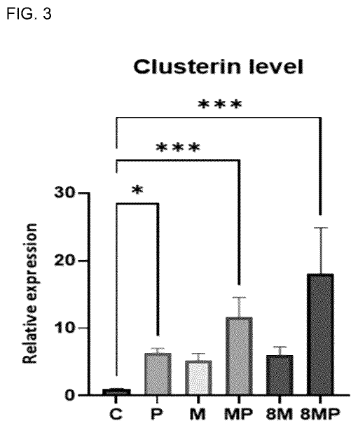

As a result, CLU expression increased in human intestinal epithelial organoids similarly to mouse intestinal epithelial organoids when treated with PGE₂ alone, and the expression was further increased in the melatonin and PGE₂ combined treatment group and 8M-PDOT and PGE₂ combined treatment group (FIG. 3).

While the present invention has been particularly described with reference to specific embodiments thereof, it is apparent that this specific description is only a preferred embodiment and that the scope of the present invention is not limited thereby to those skilled in the art. That is, the practical scope of the present invention is defined by the appended claims and their equivalents.

1. A method for preventing or treating intestinal epithelial injury disease comprising:
administering a pharmaceutical composition comprising melatonin or an agonist of melatonin receptor; and PGE₂ (prostaglandin E2) or an agonist of PGE₂ receptor as an active ingredient.

2. The method of claim 1, wherein the agonist of melatonin receptor is 8M-PDOT.

3. The method of claim 1, wherein the agonist of PGE₂ receptor is at least one selected from the group consisting of Cay10598, TCS2510, L-902,688 and ONO-AEI-329.

4. The method of claim 1, wherein the pharmaceutical composition comprises 100 to 600 μM of the melatonin or the agonist of melatonin receptor and 0.05 to 20 μM of the PGE₂ or the agonist of PGE₂ receptor.

5. The pharmaceutical composition method of claim 1, wherein the pharmaceutical composition exhibits an effect of inducing revival stem cells.

6. The pharmaceutical composition method of claim 1, wherein the pharmaceutical composition increases expression of one or more markers selected from the group consisting of clusterin, Ly6a and Claudin-4.

7. The method of claim 1, wherein the intestinal epithelial injury disease is at least one selected from the group consisting intestinal metaplasia, Crohn's disease, ulcerative colitis, ulcerative duodenitis, hemorrhagic rectal ulcer, leaky gut syndrome, gastritis, gastric ulcer, pouchitis, enteritis and ischemic colitis.

8. (canceled)

9. A composition for inducing revival stem cells comprising melatonin or an agonist of melatonin receptor; and PGE₂ (prostaglandin E2) or an agonist of PGE₂ receptor as an active ingredient.

10-11. (canceled)

12. A method of inducing revival stem cells comprising:
administering melatonin or an agonist of melatonin receptor; and PGE₂ (prostaglandin E2) or an agonist of PGE₂ receptor as an active ingredient.
